(19) United States
(12) Patent Application Publication
(10) Pub. No.: US 2025/0064089 A1
(43) Pub. Date: Feb. 27, 2025

(54) COMPOSITIONS CONTAINING CHOLINE PROPIONATE FOR CONTROLLING GROWTH OF MOLD AND IMPROVING ANIMAL PERFORMANCE AND RELATED METHODS

(71) Applicant: KEMIN INDUSTRIES, INC., DES MOINES, IA (US)

(72) Inventors: Krishna Gopal Dongol, Singapore (SG); Sai Kaw Leow, Skudai (MY); Yee Ting Wong, Singapore (SG)

(73) Assignee: KEMIN INDUSTRIES, INC., DES MOINES, IA (US)

(21) Appl. No.: 18/940,560

(22) Filed: Nov. 7, 2024

Related U.S. Application Data
(60) Provisional application No. 63/547,595, filed on Nov. 7, 2023.
(60) Provisional application No. 63/460,219, filed on Apr. 18, 2023.

(51) Int. Cl. A23K 20/105; A23K 20/179; A23K 20/195; A23K 50/75; C12Q 1/52

(52) U.S. Cl. CPC A23K 20/105; A23K 20/179; A23K 20/195; A23K 50/75; C12Q 1/52; G01N 2333/91188

(57) ABSTRACT

The present invention relates to a composition that contains choline propionate in an amount effective to controlling the growth of mold in animal feed and improve animal performance. Another aspect of the present invention relates to methods of controlling the growth of mold in animal feed, improving animal performance, and providing a protective effect on the liver. Another aspect of the present invention relates to a process for preparing a mold inhibitor composition that contains choline propionate, where the process comprises the step of using choline hydroxide as a base to neutralize propionic acid.

### CROSS-REFERENCE TO RELATED APPLICATIONS

The present application claims the benefit of priority to U.S. Provisional Patent Application No. 63/547,595, filed Nov. 7, 2023, entitled “COMPOSITIONS CONTAINING CHOLINE PROPIONATE FOR CONTROLLING GROWTH OF MOLD AND RELATED METHODS,” and U.S. Provisional Patent Application No. 63/460,219, filed Apr. 18, 2023, entitled “COMPOSITIONS CONTAINING CHOLINE PROPIONATE FOR CONTROLLING GROWTH OF MOLD AND RELATED METHODS,” the entire disclosures of which are incorporated herein by reference in their entireties.

### BACKGROUND OF THE INVENTION

Mold growth in animal feed is common during feed storage, particularly in areas of tropical temperatures and high humidity. The presence of mold in animal feed can lead to serious health consequences in animals and poultry. There are commercially available mold inhibitor products, such as Myco CURB AW Liquid (Kemin Industries, Des Moines, lowa), which contains propionic acid. In these products, the mold inhibition efficacy of organic acid has been attributed to the undissociated acid, and the efficacy increases at lower pH values. J. N. Sofos, F.F. Busta, Antimicrobial Activity of Sorbate, J. Food Prot., 44, 614-620 (1981).

However, organic acids are not without technical challenges and drawbacks. For instance, organic acids are known to have an unpleasant pungent smell and are corrosive in the presence of water. To overcome these challenges, organic acids are partially neutralized by bases, such as ammonium hydroxide or sodium hydroxide. See, e.g., Rolow et al., Corrosiveness of Liquid Mold Inhibitors Used in the Feed Industry, Mold Inhibitor Research, Report #06930/605677; Peter Golob et. al., The Use of Spices and Medicinals as Bioactive Protectants for Grains, FAO Agricultural Services Bulletin No. 37 (1999); Research and Development, Kemin Industries, Inc. “Corrosiveness of MYCO CURB A-10 Year Perspective of the Feed Industry” Mold Inhibitor General Information Report #10957; H. M. Tan, Asian Poultry Magazine, ‘Acidifiers: Synergy of acids make for better efficacy’, July 2006, 30.

Due to their volatility and highly exothermic nature, these bases require extra handling precaution during the manufacturing process. Further still, the bases currently used do not bring additional value other than neutralizing the acid, such as propionic acid, in the mold inhibitor composition.

The inventors have surprisingly found that choline hydroxide (FIG. 1) can be used as a base in mold inhibitor compositions, and unlike conventionally used bases, such as ammonium hydroxide, choline hydroxide provides superior properties to the mold inhibitor composition.

It is well documented that choline is an essential animal nutrient and plays a vital role in the synthesis of phospholipids, fats, and methyl group metabolism. Workel et al., Choline: The Rediscovered Vitamin for Poultry, The Poultry Site (2002). Phospholipids are the main constituent of the cell membrane. Phospholipids are also responsible for the emulsification of fat digestion in the gut and the transportation of nutrients in the blood vessel. It has been reported that choline has a beneficial effect in animals and humans in controlling fatty liver syndrome and preventing hemorrhagic kidneys. Saeed et al., Journal of Experimental Biology and Agriculture Science, Beneficial Impact of Choline in Animal and Human, 5, 5,589-598 (2017).

Choline chloride is the most commonly used source of choline in animal feed. Depending on the application, choline chloride may be added directly into the animal feed or via a premix into the feed mash. For poultry and swine diets, for instance, the recommended dosages of choline chloride are 250-700 mg/kg feed and 250-600 mg/kg feed, respectively.

Several choline ionic liquids can be synthesized by reacting choline base with organic acids. Tanzi L. et al., Structure Study on Choline-carboxylate Bio-ionic Liquid by X-ray Scattering and Molecular Dynamics, J. Chem. Phys, 143, 114506 (2015). By reacting choline hydroxide with propionic acid, a choline salt possessing ionic liquid properties will be formed (FIG. 2). Choline ionic liquids have been reported to have benefits such as higher surface activities, lower vapor pressure, and melting point making the new organic choline derivatives more favorable over inorganic choline chloride in the application as feed additives or premix, especially in countries with cold weather. Smith et al., Deep Eutectic (DESs) and Their Application, Chem. Rev. 114, 21, 11060-11082 (2014).

The inventors have surprisingly discovered that using choline hydroxide to neutralize organic acids, such as propionic acid, results in a composition that is suitable for administration to animals, for instance as a feed additive.

### BRIEF SUMMARY OF THE INVENTION

The present invention relates to a composition that contains choline propionate in an amount effective to controlling the growth of mold in animal feed. Another aspect of the present invention relates to methods of controlling the growth of mold in animal feed by adding a composition that contains choline propionate to the animal feed. Another aspect of the present invention relates to a process for preparing a mold inhibitor composition that contains choline propionate, where the process comprises the step of using choline hydroxide as a base to neutralize an organic acid, for instance propionic acid, where the resulting composition exhibits superior properties, such as mold inhibition, and surprisingly reduced corrosivity on the feed mill equipment. Additionally, another aspect of the present invention relates to administering a choline salt, such as choline propionate, as a feed additive in an amount that inhibits mold in feed and provides additional nutritional benefit to the animal.

According to at least one embodiment, the feed additive or supplement resulted in improved animal production, such as improved broiler performance, which can be measured for instance by an increased weight gain. According to another embodiment, the nutritional benefit or animal production includes, for instance in laying hens, increased egg production and improved egg quality. For instance, in at least one embodiment, the nutritional benefit includes, for instance in laying hens, increased egg production and improved egg quality, for instance increase in yolk weight and/or desired yolk color. In at least one embodiment, the compositions of the present invention confer a protective effect of the liver as measured by blood serum analysis.

In at least one aspect, the present invention relates to a feed additive or supplement that is capable of controlling the growth of mold in the animal feed and providing a nutritional benefit to the animal. In at least one embodiment, the nutritional benefit to the animal is measured by improved animal performance. In another aspect, the inventors have identified methods of administering a composition containing choline propionate to animals by adding the composition to the animal's diet, where adding the composition to the feed inhibits the growth of mold on the feed and enhances the animal performance when it is consumed. In certain embodiments, animal performance can be measured in broilers by improvements in body weight gain, feed intake, feed conversion ratio or production efficiency index, and in laying hens by improvements in egg production and egg quality, including but not limited to an increase in yolk weight or desired yolk color.

In certain embodiments, the composition includes choline propionate in combination with other components, such as surfactants, co-surfactants, preservatives, pigments, colorants, flavoring, anti-microbial, antioxidant, or other additives.

### BRIEF DESCRIPTION OF THE FIGURES

FIG. 1 is a drawing of choline hydroxide.

FIG. 2 is a drawing of a choline salt.

FIG. 3 is a chart that compares free acid (%) and pH with varying concentrations of choline hydroxide.

FIG. 4 depicts the CO₂ evolution (%) of the Control (MCL AW) vs AW14 1 kg/ton, moisture 12.06%, a_{w}: 0.789, at room temperature.

FIG. 5A depicts the comparison of mold inhibition of AW14 with the Control (MC AWL).

FIG. 5B depicts the comparison of mold inhibition of AW14 with the Control (MC AWL).

FIG. 6 depicts the metal corrosivity test of MCL AW and AW 40.

FIG. 7 depicts the CO₂ evolution (%) of MCL AW vs AW40 at moisture 15.1%, a_{w}: 0.876, room temperature (25° C.), 1 kg/ton.

FIG. 8 depicts the CO₂ test comparison of MCL AW (AW) vs. AW40 at 14% moisture content, room temperature (25° C.) at 0.5, 1.0 and 2.0 kg/ton.

FIG. 9 depicts the CO₂ test comparison of MCL AWL (AW) vs AW40 at moisture 15%, room temperature at 0.5, 0.75 and 1.0 kg/ton.

FIG. 10A compares the mold inhibition by ZOI of AW40 with MCL AW.

FIG. 10B compares the mold inhibition by ZOI of AW40 with MCL AW.

FIG. 11A shows the results of the metal corrosivity test of MCL AW and AW40.

FIG. 11B shows the results of the metal corrosivity test of MCL AW.

FIG. 11C shows the results of the metal corrosivity test of AW40.

FIG. 12 compares the surface tension of MCL AW and AW14.

FIG. 13 depicts the reaction scheme of choline hydroxide and propionic acid.

FIG. 14 is the FTIR spectrum of propionic acid, choline hydroxide, and choline propionate.

FIG. 15 is the ¹H NMR of choline propionate.

FIG. 16A summarizes the milling trial results.

FIG. 16B summarizes the milling trial results.

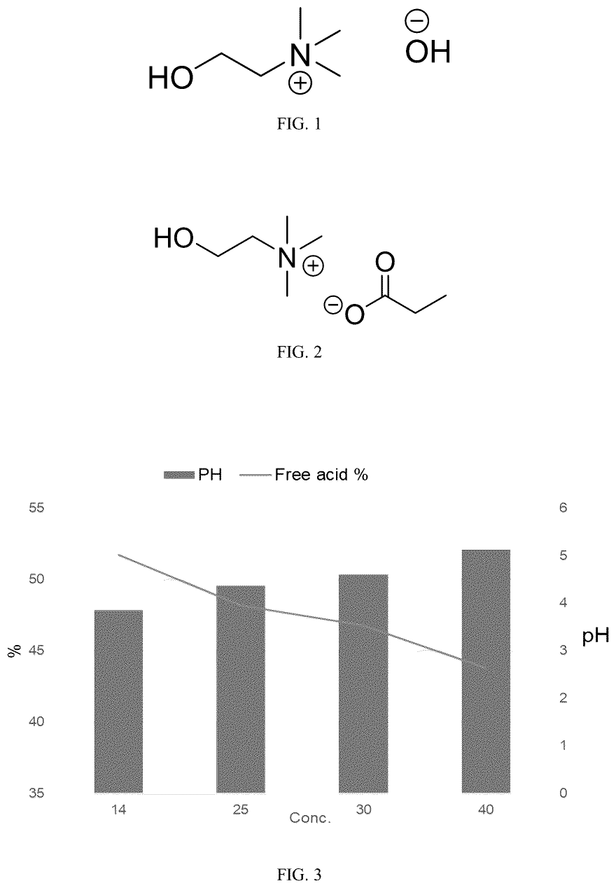

### DETAILED SUMMARY OF THE INVENTION

The present invention relates to a composition that contains choline propionate in an amount effective to controlling the growth of mold in animal feed. Another aspect of the present invention relates to methods of controlling the growth of mold in animal feed by adding a composition that contains choline propionate to the animal feed. Another aspect of the present invention relates to a process for preparing a mold inhibitor composition that contains choline propionate, where the process comprises the step of using choline hydroxide as a base to neutralize an organic acid, for instance propionic acid, where the resulting composition exhibits superior properties, such as mold inhibition, and reduced corrosivity.

The present invention relates to a composition that contains choline propionate in an amount effective to controlling the growth of mold in animal feed. Another aspect of the present invention relates to methods of controlling the growth of mold in animal feed by adding a composition that contains choline propionate to the animal feed. Another aspect of the present invention relates to a process for preparing a mold inhibitor composition that contains choline propionate, where the process comprises the step of using choline hydroxide as a base to neutralize an organic acid, for instance propionic acid, where the resulting composition exhibits superior properties, such as mold inhibition, and surprisingly reduced corrosivity on the feed mill equipment. Additionally, another aspect of the present invention relates to administering a choline salt, such as choline propionate, as a feed additive in an amount that inhibits mold in feed and provides additional nutritional benefit to the animal. According to at least one embodiment, the feed additive or supplement resulted in improved animal production, such as improved broiler performance, which can be measured for instance by an increased weight gain. According to another embodiment, the nutritional benefit or animal production includes, for instance in laying hens, increased egg production and improved egg quality. According to another embodiment, the compositions of the present invention provide a protective effect of the liver.

In at least one embodiment of the present invention, the composition includes choline propionate in an amount effective to inhibit the growth of mold in animal feed. In certain embodiments, the composition includes choline propionate in combination with other components, such as surfactants, co-surfactants, preservatives, pigments, colorants, flavoring, anti-microbial, antioxidant, or other additives. In certain embodiments, the composition includes a surfactant or one or more surfactants or blends.

In certain embodiments, the composition may optionally include one or more essential oils. By way of non-limiting example, the essential oil includes but is not limited to cinnamon bark oil, oregano oil and thyme oil.

According to at least one embodiment, the present invention is a mitigation measure to control and/or to prevent the growth of mold in animal feed by adding a composition that contains choline propionate. In at least one embodiment, the composition is added to the feed. In certain embodiments, the composition is applied or sprayed onto the feed. In certain embodiments, the composition is added to the feed using various applications and known technologies.

According to at least one embodiment, the present invention is an animal feed supplement or additive that is capable of controlling mold growth and provides improved animal performance. In certain embodiments, animal performance can be measured by improvements in body weight gain, feed intake, feed conversion ratio or production efficiency index. For example, in broilers, animal performance can be measured by improvements in body weight gain, feed intake, feed conversion ratio or production efficiency index. For example, in laying hens, animal performance can be measured by improvements in egg production and egg quality, including but not limited to an increase in yolk weight or desired yolk color.

In at least one aspect, the present invention relates to a feed additive or supplement that is capable of controlling the growth of mold in the animal feed and providing a nutritional benefit to the animal. In at least one embodiment, the nutritional benefit is demonstrated by improvements in body weight gain, feed intake, feed conversion ratio or production efficiency index, egg production and egg quality, including but not limited to an increase in yolk weight or desired yolk color.

According to at least one embodiment, the compositions of the present invention are suitable for animal feed and can be combined with known animal feed ingredients, including but not limited to corn meal, soybean meal, fish meal, soy oil cake, dried distillers' grains with solubles (DDGS), etc.

For purposes of this disclosure, “animal feed” refers to corn & soybean meal provided to livestock. The animal feed may be provided to the animals through any convention means well known to persons skilled in the art, include but not limited to top-dress, mixed in by hand, pelleted, mixed with crumbles, etc.

### EXAMPLES

### Example 1

The researchers investigated the use of choline hydroxide as a base to replace ammonium hydroxide as a neutralizing agent. Several prototypes (AW14, AW25, AW30, and AW40) with varying concentrations of choline content were prepared. All prototypes had higher free acid content and higher corrosivity than the control (MCL AW) except AW 40, which showed less corrosivity than MCL AW, even though it has a higher free acid content. From the CO2 evolution and zone of inhibition (ZOI) tests, the AW 40 prototype demonstrated better mold inhibition efficacy. AW 40 also showed lower surface tension than MCL AW, which potentially could improve milling efficiency. To confirm the formation of choline propionate in AW 40, choline propionate was synthesized by the reacting 1:1 mole ratio of choline hydroxide and propionic acid. The choline product was characterized using FTIR and 1HNMR analysis.

### Materials and Methods

Materials. Ammonium hydroxide, propionic acid, sorbic acid dry, phosphoric acid, Tween 80 liquid, polyalcohol dibase IV and Choline hydroxide (CAS 123-41-1) were purchased from Sigma-Aldrich and corn feed was obtained from N&N Agriculture Pte Ltd, Singapore. Myco CURB (“MCL AW”) was used as the positive control for purposes of this study. The compositions for the prototypes are summarized in Table 1.

TABLE 1
Formulation details of Control (MCL AW) and prototypes (AW14,
AW25, AW30, and AW40) with choline hydroxide.

| | | MCL AW | AW14 | AW25 | AW30 | AW40 |
|---|---|---|---|---|---|---|
| RM Code | Item Name | % | % | % | % | % |
| RM05136 | Sorbic acid dry | 0.297 | 0.297 | 0.297 | 0.297 | 0.297 |
| RM05650 | Phosphoric acid 85% | 0.495 | 0.495 | 0.495 | 0.495 | 0.495 |
| | Liquid | | | | | |
| RM05656 | Propionic acid liquid | 55.4 | 55.4 | 55.4 | 55.4 | 55.4 |
| | 99% | | | | | |
| RM05594 | Kemin AA 25% Liq* | 24.59 | 0 | 0 | 0 | 0 |
| | DI water | 17.08 | 28.01 | 16.67 | 11.67 | 0 |
| RM05687 | Tween 80 Liq | 1.98 | 1.98 | 1.98 | 1.98 | 1.98 |
| CAS 123- | Choline hydroxide 45% | 0 | 13.66 | 25.00 | 30.00 | 41.67 |
| 41-1 | | | | | | |
| 829078 | Polyalcohol Dibase IV | 0.158 | 0.158 | 0.158 | 0.158 | 0.158 |
| | Total | 100 | 100 | 100 | 100 | 100 |

*Kemin AA 25 Liq contains 25% ammonium hydroxide.
AW14 = 1:1 wt. % replacement of ammonium hydroxide with choline hydroxide (13.66% choline hydroxide);
AW25 = 25% choline hydroxide;
AW30 = 30% choline hydroxide;
AW40 = equimolar replacement of ammonium hydroxide with choline hydroxide (41.67% choline hydroxide).

The weight of each ingredient was first calculated according to their composition in each prototype. In a glass bottle, weighed amounts of chemicals were added with the addition sequence of sorbic acid, phosphoric acid, propionic acid, Kemin AA 25 liquid/choline hydroxide, polyalcohol dibase, glycerol and tween-80. The prototypes were stirred to obtain a homogenous solution.

Determination of moisture. The moisture of the feed was determined using the Heraeus Instruments Drying Oven. About 2 g of the feed was weighed onto a pre-weighed aluminum plate and the initial weight of the plate was recorded. The plate was then placed in an oven at 135° C. for 2 hours. After 2 hours, the aluminum plate was placed in the desiccator for 30 minutes, after which the weight was taken. This test was performed in triplicates and the average results were reported. Moisture content can then be calculated with Equation 1:

$$Moisture determination ( \% )$$

$$\% Moisture content = \frac{W 2 - W 3}{W 1} \times 100 \qquad Equation 1$$

where W1=weight of the sample,
W2=total weight of pan+sample before 2 hours at 135° C.,
W3=final weight of pan+sample after 2 hours at 135° C.

Adjustment of moisture content. The desired moisture content of the feed was obtained by using Equation 2.

$$Moisture adjustment ( g )$$

$$Water added in g = \frac{( Final moisture context \times Feed quanity in g ) - ( 100 \times Initial moisture content in feed ) )}{( 100 - Final moisture content )} \qquad Equation 2$$

An appropriate amount of deionized water was added to the known quantity of feed. To ensure homogeneity, the Kenwood Major Classic Mixer Model KM800 was used for mixing purposes. The feed was kept in a sealed bag and placed in a refrigerator of 2-8° C. overnight. The feed was thawed to room temperature and the adjusted moisture content was determined before use.

Fourier-transform infrared spectroscopy (FTIR) analysis. The molecular structure and functional groups of choline propionate were characterized by the FTIR instrument, PerkinElmer FTIR Spectrum Two, with 16 scans.

Determination of water activity (a_{w}). Water activity in the feed is used to describe the amount of unbound water available for microbiological growth. The water activity of the corn grains was analyzed using the AquaLab water activity meter.

Mixing of feed with mold inhibitors and CO₂ test. Corn feed was ground to mash form and the treated samples were prepared as follows: In a zip lock bag, 0.3 g of the mold inhibitor (MC AWL or the choline prototypes) was added to 300 g of feed and mixed for 5 minutes. (treatment was equivalent to 1 kg of dry mold inhibitor per ton of feed). In a carbon dioxide bottle (500 mL glass bottle with fabricated cap), 150 g of feed was then loaded, and the cap was closed tightly. The carbon dioxide bottles were incubated at 25° C., and the carbon dioxide content of the treated and untreated feed was measured daily using the Edinburgh Sensors-Guardian Plus model no. D500.

Zone of inhibition (ZOI) test. All chemicals and reagents used were of analytical grade. The assay of the propionic acid and its content in the prototypes were determined by an acid-base titration method of Kemin Asia (WI-QCR-003). The efficacy of the mold inhibitor formulations was tested by a plate diffusion test using two stains for the mold inhibition study (WI-KAI-003), for Aspergillus favus and Penicillium chrysogenum.

Corrositivity test. Galvanized iron strips (Scientific Technical Supplies Pte Ltd, Singapore) with dimensions of 3.5 cm by 7 cm were used in all the tests. The specification of the strips was galvanized metal hot dipped JIS G33022 Z22 (SGCC). The metal strips were washed carefully with detergent and rinsed ten times with tap water, five times with de-ionized water and technical grade acetone to remove dirt and grease. The bottles and strips were dried in an oven at 105° C. for two hours. The strips were then cooled in a desiccator. Each test strip was weighed and then immersed 3.5 cm deep into approximately 66 g of MCL AW and the AW prototypes in a 175 ml dried glass bottle. All the bottles were incubated at 65° C. for 11 days. After 11 days, these strips were carefully removed from the bottles without disturbing the rusted metal surface. The strips were cleaned using ultra-sonication (Leo-150, Acoustical Technologies(S) P/L) for 1 hour. The metal strips were then washed again as described above. The loss in metal strip weights was then measured.

### Results

### Properties of AW Prototypes at Different Choline Hydroxide Neutralization Formulation

The various prototypes were formulated by neutralizing choline hydroxide from 1:1 wt. % to 1:1 mole ratio replacement of ammonium hydroxide, which is used in the preparation of the commercially available Myco CURB AW Liquid (MCL AW) formulation. The prototypes were named as: AW 14, AW 25, AW 30 and AW 40 by neutralizing choline hydroxide at 13.7%, 25.0%, 30.0% and 41.7% respectively. The appearance (FIG. 3), pH, and free acid content were analyzed, and the water content and choline propionate calculation are summarized in Table 2.

TABLE 2
Properties of the samples

| | | | | Moisture | Choline |
|---|---|---|---|---|---|
| | | | Free Acid | content | propionate, |
| Samples | Appearance | pH | % | % | mg/kg* |
| MCL AW | Dark purple | 5.26 | 41.6 | 35.52 | 0 |
| AW14 | Golden yellow | 3.86 | 51.7 | 35.52 | 89.9 |
| AW25 | Golden yellow | 4.36 | 48.2 | 30.42 | 164.5 |
| AW30 | Golden yellow | 4.60 | 46.8 | 28.17 | 197.5 |
| AW40 | Golden yellow | 5.13 | 43.8 | 22.92 | 274.2 |

*theoretical calculation.

With an increasing amount of choline hydroxide in the AW prototype formulations, the free acid values (%) decreased, and pH increased as shown in FIG. 3.

With the increase content of choline hydroxide, the pH decreases proportionally, indicating salt formation in the mixture. Decreasing pH and free acid by adding choline hydroxide also proved that choline hydroxide can be used as an alternative to ammonium hydroxide in buffering the organic acid.

Myco CURB AW liquid prototype formulated using choline hydroxide in 1:1 weight % ammonium hydroxide. A new prototype AW 14 was formulated by neutralizing the propionic acid with choline hydroxide in 1:1 weight % of ammonium hydroxide in Myco CURB AW liquid. The mold inhibition efficacy of AW 14 was compared against MCL AW using carbon dioxide evolution (CO₂) and zone of inhibition (ZOI) tests.

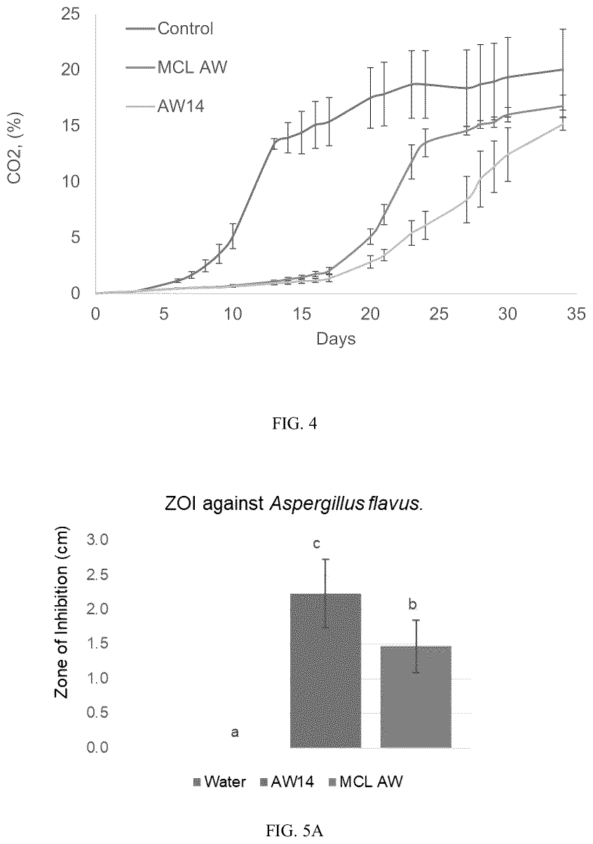

Carbon dioxide evolution (CO₂) Test. The comparison of the efficacy of mold inhibition of MCL AW and AW 14 was performed by mixing the mold inhibitors (AW 14 and MCL AW) at 1 kg/ton with mash corn feed with 12.06% moisture (a_{w}: 0.789). At room temperature, the accumulation of CO₂ was measured for each treatment group, and CO₂ evolution for 35 days is shown in FIG. 4.

In the first CO₂ study, AW 14 with choline salt showed better mold inhibition efficacy than Myco CURB AW.

Zone of inhibition (ZOI) test. The zone of inhibition was compared of AW 14 and MCL AW using two species of mold, Aspergillus flavus and Penicillium chrysogenum. The inhibition zone results are shown in FIG. 5.

In the ZOI study of AW 14 and MCL AW for Aspergillus flavus and Penicillium chrysogenum, AW 14 showed a bigger ZOI than MCL AW. The higher free acid content could explain the higher ZOI in AW 14 as compared to MCL AW. The CO₂ and ZOI analysis showed that AW 14 showed higher mold inhibition efficacy than MCL AW. However, the free acid analysis of AW 14 showed higher free acid content than MCL AW.

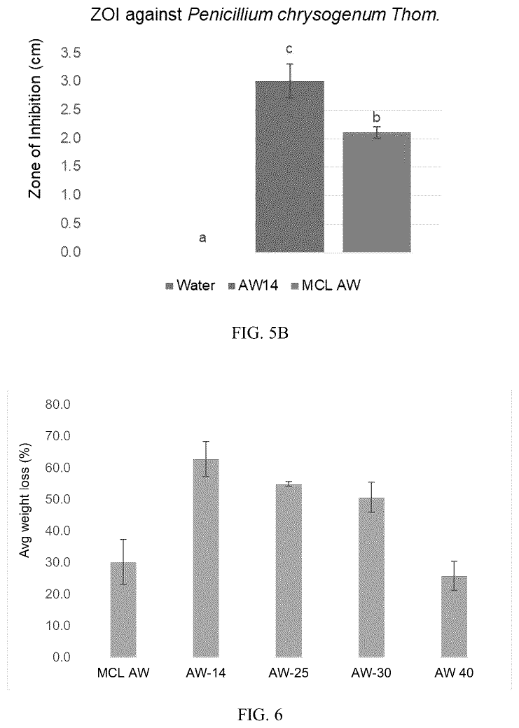

Corrosivity test: The corrosivity test of prototypes AW 14, AW 25, AW 30, and AW 40 was done using galvanized iron strips with the dimension 3.5 cm by 7 cm. The result of corrosivity is shown in FIG. 6.

The researchers observed that the corrosivity effect reduced with a decreasing free acid content in AW 14, AW25, AW30, and AW40. Even though the corrosivity decreased with the increase of choline hydroxide, AW 40 demonstrated the lowest (25.8% metal loss) and MCL AW 14 recorded the highest (32.4% metal loss) corrosivity. Unexpectedly, AW 40 had a lower corrosivity than MCL AW even though the free acid content of AW 40 (43.8%) was higher than MCL AW (41.6%). In view of this, AW 40 was selected for further study for mold efficacy, surface tension activity and characterization.

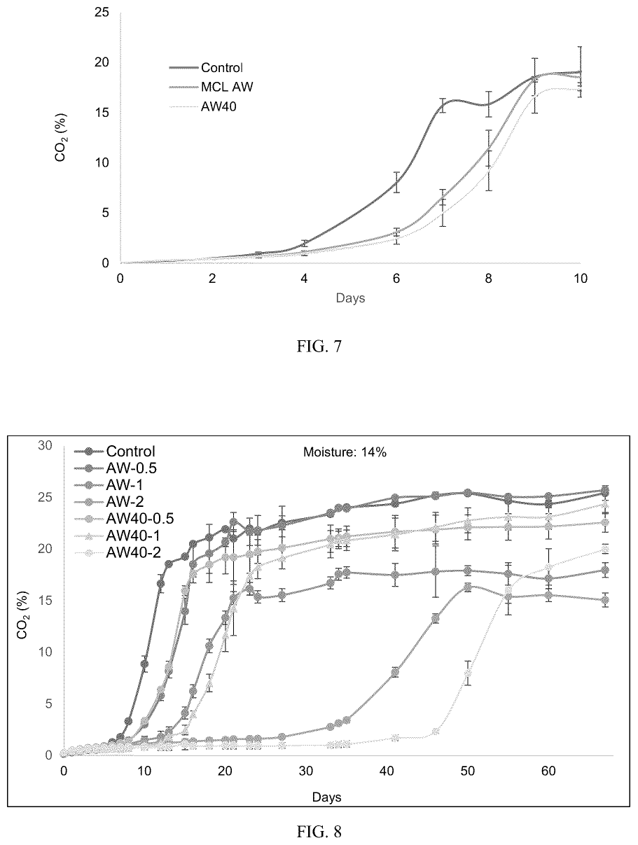

Formulation of prototype AW 40. Further studies using the new prototype, AW 40 was formulated by neutralizing the propionic acid with choline hydroxide in a 1:1 molar ratio of ammonium hydroxide in Myco CURB AW liquid. Based on the formulation of Myco CURB AW liquid, the maximum inclusion level of choline hydroxide was 41.67% (AW 40). This is equivalent to a 1:0.85 molar replacement of ammonium hydroxide in the formulation. The mold inhibition efficacy of MCL AW and AW 40 was performed by mixing the mold inhibitors with mash corn feed with 14.1% moisture (a_{w}: 0.876) at 1 kg/ton. Another treatment group at higher (15%) moisture was used to accelerate the result for two weeks. The study showed that AW40 demonstrated numerically better mold inhibition efficacy than MCL AW. (FIG. 7)

CO₂ Dose Dependent Test: The AW40 and Myco CURB AW liquid (MCL AW) were selected to study the dose response effect on mold inhibition. The CO₂ evolution test was conducted at four doses, 0.5 kg/t, 1.0 kg/t, 2.0 kg/t and 3 kg/t in triplicates at 14% moisture, 25° C. The result of CO₂ tests up to 70 days is shown in FIG. 8. Since no mold growth was observed at 3 kg/t for 100 days, the CO₂ curved was not included in the below graph (FIG. 8).

The difference in CO₂ induction point increased with the increase of treatment dose from 0.5 to 2 kg/t of AW 40 and MCL AW, as shown in FIG. 8.

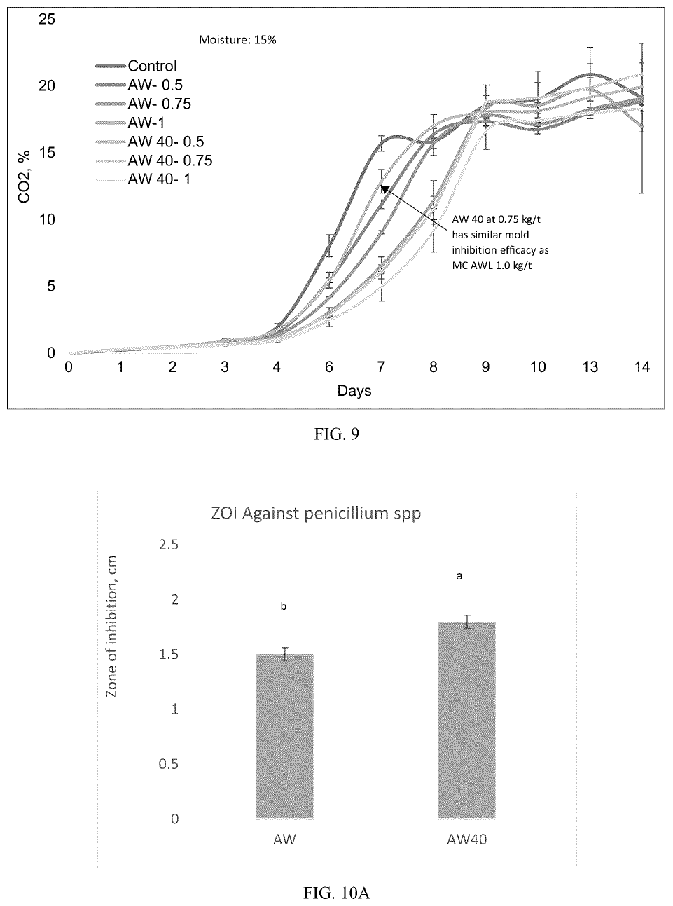

In another CO₂ dose effect test of AW 40 and MCL AW at three doses: 0.5 kg/t, 0.75 kg/t, and 1.0 kg/t in triplicates at 15% moisture (accelerated), 25° C., no difference in mold inhibition efficacy at 0.5 kg/t was observed between AW 40 and MCL AW. However, AW40 at 0.75 kg/t showed similar mold inhibition efficacy as MCL AW at 1 kg/t (FIG. 9).

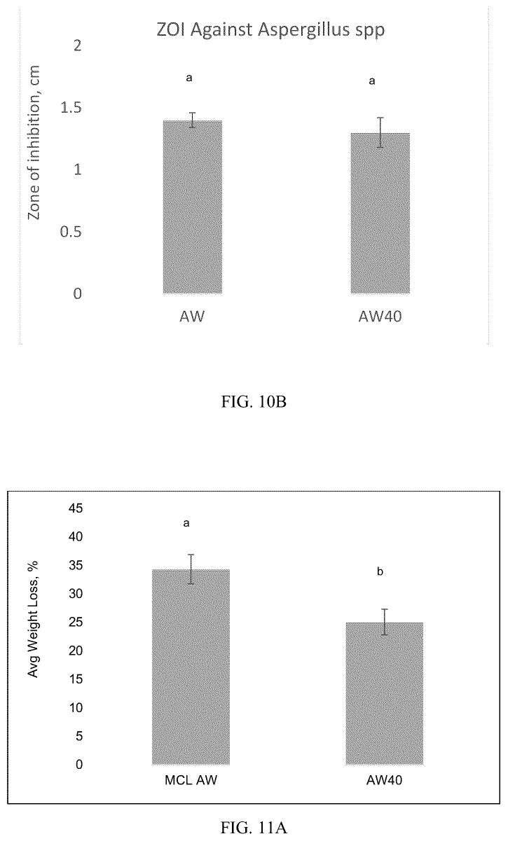

The ZOI of AW40 was then compared against MCL AW for Aspergillus flavus and Penicillium chrysogenum. Interestingly, there was no significant difference in the inhibition of aspergillus despite the different amounts of free acid content in MCL AW and AW40. However, AW 40 showed significantly higher inhibition of penicillium than MCL AW FIG. 10.

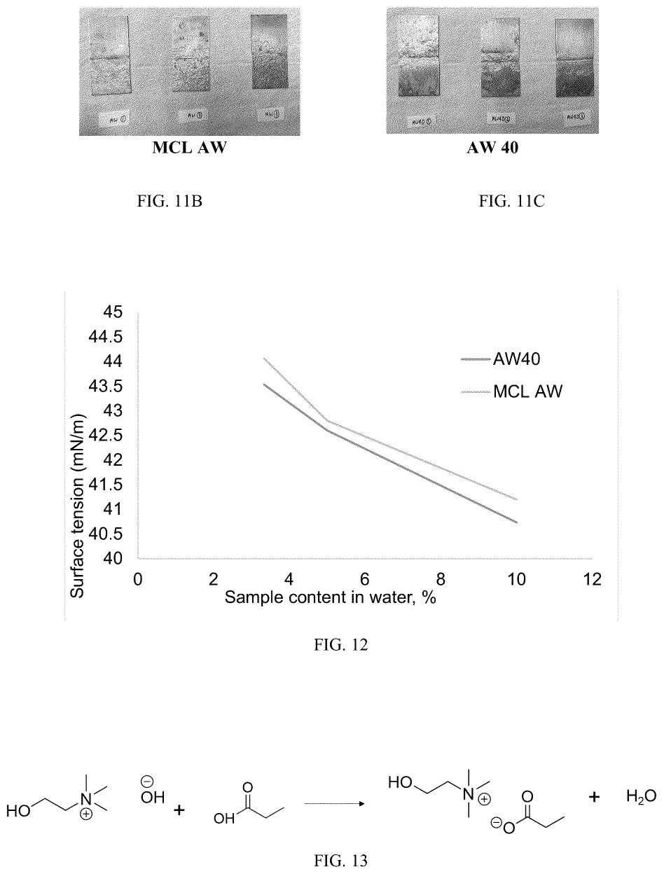

Corrosivity of AW40 vs. MCL AW. The corrosivity test of prototypes AW40 and MCL AW were done using galvanized iron strips with the dimension 3.5 cm by 7 cm. the result of corrosivity and pictures of metal strips after 11 days are shown in FIG. 11.

AW40 showed lower corrosivity than MCL AW even though the free acid content of AW40 (43.8%) is higher than MCL AW (41.6%) which is a similar result to a previous corrosivity test. These unexpected results could be explained due to ionic liquid properties of AW40 which will lower the vapor pressure of acid and water molecules.

Surface tension of AW40 vs MCL AW. Surface tension was considered to investigate the effect of choline propionate on the AW40 and MCL AW; the result is shown in FIG. 12.

The surface tension of AW40 was lower than MCL AW. This result implied that AW40 has higher wettability and spreadability properties than MCL AW which could an advantage during the feed milling process.

Characterization of choline propionate. With the increase in the neutralization of propionic acid by choline hydroxide, the free acid content decreases and increases the pH of the system (FIG. 3). To confirm the formation of choline propionate from the neutralization of choline hydroxide (45%) with propionic acid (99%) in the MCL AW formulation, choline propionate was separately prepared by a complete neutralization of propionic acid with choline hydroxide. The defined weight of propionic acid was taken into the round bottom flask and then neutralized in a 1:1 mol ratio by choline hydroxide (FIG. 13). The mixture was stirred at room temperature for 24 hours, and water was evaporated using a rotary vacuum at 95° C.

TABLE 3
Preparation of choline propionate

| | Choline | Propionic | Choline | |
|---|---|---|---|---|
| | hydroxide, | acid | propionate | Water |
| Molecular | 104.17 | 74.08 | 177.24 | 18 |
| Weight | | | | |
| Molar | 0.1 | 0.1 | 0.1 | 0.1 |
| Weight (g) | 26.93 | 7.41 | | |
| Weight % | 78.51 | 21.55 | | |

To compare the raw materials and product spectrum, the FTIR and 1H-NMR analyses were done for choline hydroxide, propionic acid, and choline propionate.

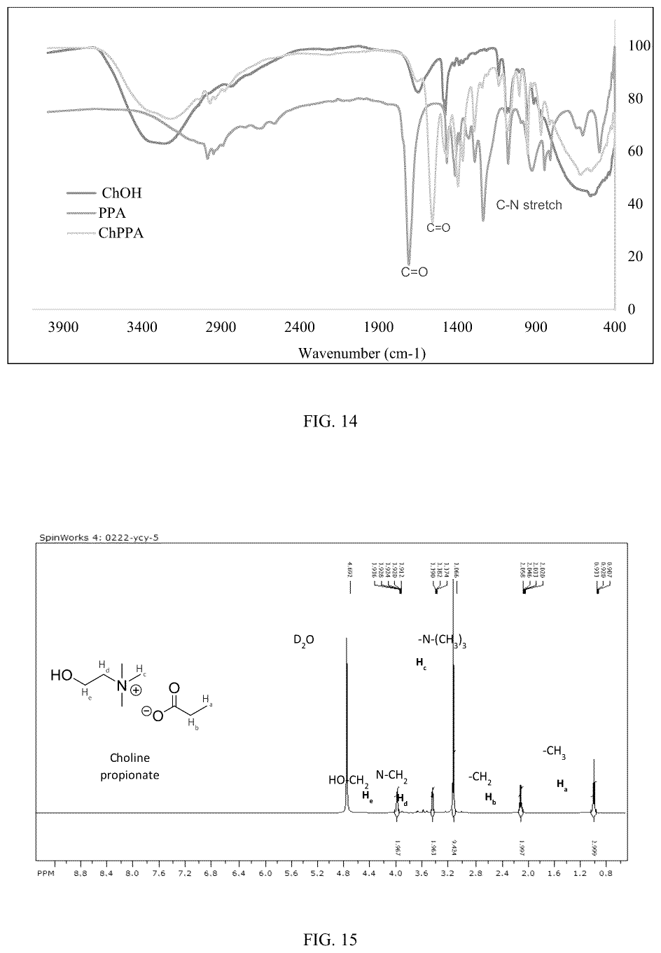

The FTIR spectrum of the three samples are shown in FIG. 14. The sharp carbonyl (C=0) peak of propionic acid at 1707 cm⁻¹ shifted to 1558 cm⁻¹ after the reaction with choline hydroxide. This could be explained by hydrogen bond formation between ammonium cation and propionate anion forming a choline propionate salt. The tert-amine peak at 1642 cm⁻¹ and O—H stretching peak at 3271 cm⁻¹ of choline hydroxide shifted to 1550 cm⁻¹ and 3371 cm⁻¹ respectively at the choline propionate FTIR spectrum. The shift and broadening in peaks confirmed the formation of choline propionate.

¹H NMR further identified the choline propionate. The comparative ¹HNMR spectrums shift of the choline hydroxide, propionic acid, and choline propionates are shown in Table 4.

TABLE 4
The chemical shift of choline Hydroxide,
propionic acid and choline propionate

| | Choline | | |
|---|---|---|---|
| δ | Hydroxide | Propionic acid | Choline propionate |
| Hₐ | — | 0.890 | 0.920 |
| H_{b} | — | 2.200 | 2.040 |
| H_{c} | 3.056 | — | 3.066 |
| H_{d} | 3.305 | — | 3.382 |
| Hₑ | 3.889 | — | 3.924 |

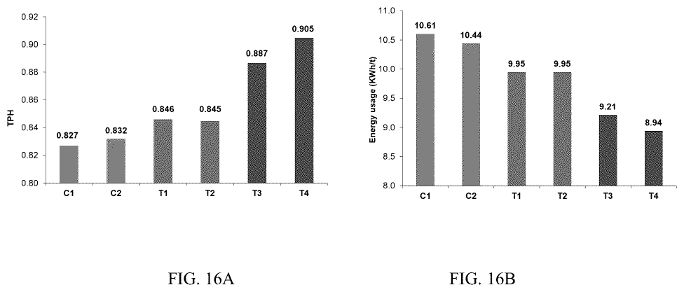

The peaks of propionic acid at 0.890 and 2.200 ppm shifted to 0.920, 2.040 ppm in the choline propionate compound. The choline hydroxide peaks at 3.056, 3.305 and 3.889 ppm shifted to 3.066, 3.382 and 3.924 ppm in the synthesized prototype confirmed the change of chemical environment of a new choline propionate molecule. Each peak of the choline propionate compound was identified in the ¹HNMR spectrum as shown in the FIG. 15.

### Results

The researchers surprisingly found that choline hydroxide is a promising alternative to ammonium hydroxide in the preparation of a mold inhibitor composition. In this study, the researchers determined that the mold inhibition efficacy of AW40 neutralized by choline hydroxide was comparable to the control (MCL AW). On a 1:0.85 mole replacement of ammonium hydroxide with choline hydroxide, AW 40 demonstrated better mold inhibitory efficacy than MCL AW with lower corrosivity although it contained higher free acid content. With higher concentrations of choline hydroxide in prototypes AW25, AW30 and AW40, the pH increases, and free acid decrease showing the formation of choline salt. The formation of the choline propionate salt was confirmed using FTIR and ¹HNMR analysis. The new prototype AW40 demonstrated lower surface tension which is an additional beneficial property in the milling process. AW40 contained 160 ppm choline, which has the useful potential to partially replace the conventional use of choline chloride in feed applications, which is hygroscopic and can be difficult to handle.

### Example 2

### Materials and Methods

The basal diet was divided into 4 blocks, each block with 1,200 kg basal mash feed. Six treatment diets, 200 kg per diet, were prepared for each block with the test product supplementation and added water as summarized in Table 5.

TABLE 5
Summary of control diet and treatments

| Treatment | Diet description and product application |
|---|---|
| C1 | Basal diet + 1 kg *Dry mold inhibitor product |
| | containing 35% propionic acid (no water) |
| C2 | Basal diet + 1 kg *Dry mold inhibitor product |
| | containing 35% propionic acid + water 1% |
| T1 | Basal diet + *MCL AW 0.75 kg/t + water 1% |
| T2 | Basal diet + *MCL AW 1.00 kg/t + water 1% |
| T3 | Basal diet + *MCL AW-40 0.75 kg/t + water 1% |
| T4 | Basal diet + *MCL AW-40 1.00 kg/t + water 1% |

Each treatment diet was mixed thoroughly by a 200-kg double ribbon type mixer. The ready mixed 200 kg mash feed of each treatment within each block was processed through a steam conditioner (85°° C. conditioning temperature, 30 seconds retention time) and through 3-mm pellet die holes before being conveyed to a pellet cooler (cooling time of approximately 30 minutes).

As shown in FIG. 16, the researchers observed that both LP1-0.75 W and LP1-1.00 W treatments exhibited better milling efficiencies when compared with current MCAw by increasing production efficiency, tonne per hour (TPH) and reduce energy usage. This could be due to the mode of action in AW 40 solution creating a better lubricant effect to lower pellet mill motor load and lower electrical energy usage. The researchers hypothesized that the choline propionate could be providing a lubricant effect.

### Example 3

### Materials and Methods

The prototype AW40 containing 19% of choline hydroxide was prepared for this study. Growth performance was carried out at different growth phases from Day 1 to 35 days. Additional evaluation on carcass traits, cut yields, fatty liver assessment and preliminary gut microbiota measurement were included as part of the study.

Birds, facilities and management. The trial was conducted by Animal Research Consultant (ARC) at NKP Research Farm in Nikompattana District of Rayong Province, Thailand. A total of 640 newly hatched male broiler chicks of commercial strain (Ross 308) were used in this study. Birds were allocated to 64 pens with 10 birds per pen (normal stocking density). Eight Treatment Groups (T1, T2, T3, T4, T5, T6, T7 and T8) were randomly allocated to the birds, each treatment has 8 replicates per pen. Each Treatment Group had 80 birds. The experiment was conducted in a close house with tunnel ventilation and evaporative cooling system. Birds were raised on concrete floor pens (1 m×1 m) using rice hull as bedding material and had free access to feed and water. Each pen was set with a tubular feeder and 2 nipple water drinkers. Birds received vaccination against ND (Newcastle Disease) and IB (infectious bronchitis (IB) on day 7 and IBD (Infectious bursal disease) on day 14. Other management systems were conducted according to the recommended guideline for Ross 308 management manual.

Diet preparation. The diets were provided to birds in three phases, starter (0-10 day), grower (10-24 day) and finisher (24-35 day). In Study 1, a Corn-Soy Bean Meal (CSBM)-based, F1 diet was formulated with all nutrients meeting requirements except with insufficient choline content (no added choline). For Study 2, a second feed diet which is CSBM-based, F2 was formulated with the same nutrient levels as F1 but with added choline chloride to meet requirements but with 20% reduced Methionine and Cysteine (with no added DL-Met). The composition and calculated nutrient contents are presented in Tables 2, 3 and 4. The test products, MCL AW, choline chloride and AW40 were added in the basal diets as shown in the treatment experimental design. The choline content in each of the formulated diets for Study 1 and 2 is tabulated in Table A (Supplementary Section). Feeds were prepared in crumble form for all birds during the first 10 days and in pellet form thereafter until finishing the 35-day test period.

Experimental Design. The study was conducted in two groups, Study 1 and Study 2. In both studies, choline chloride was also included for comparison against efficacy from choline propionate in AW40 in Treatment Groups, T2, and T6.

Study 1: Feed F1 with a Choline-deficient Diet
T1: Control with MCL AW at 1.0 kg/T
T2: Choline chloride with MCL AW at 1.0 kg/T
T3: Choline propionate in AW40 at 0.75 kg/T
T4: Choline propionate in AW40 at 1.0 kg/T
   Study 2: Feed F2 with Methionine-reduced Diet (Reduced M+C by 20%)
T5: Control with MCL AW at 1.0 kg/T
T6: Choline chloride with MCL AW at 1.0 kg/T
T7: Choline propionate in AW40 at 0.75 kg/T
T8: Choline propionate in AW40 at 1.0 kg/T

The added choline content from AW40 in T4 was equivalent to the choline content from choline chloride of T2. The amount of propionic acid in AW40 in T4 was equivalent to that in MCL AW of T1 and T2.

The added choline from AW40 in T8 was equivalent to the choline from choline chloride of T6. The amount of propionic acid in AW40 in T8 was equivalent to MCL AW of T5 and T6. AW40 treatment dosage at 0.75 kg/T and 1.0 kg/T were used according to the usual recommendations for KAA customers based on existing mold inhibitor products.

Growth Performance Evaluation. Body weight gain, feed intake, feed conversion ratio (FCR) and production efficiency index were taken for the period of 0-10, 24-35, 0-24 and 0-35 days of age. Body weight and feed consumption were measured based on per pen basis at the beginning and the end of each feeding phase (0, 10, 24 and 35 of age). Number and weight of dead and culled birds were recorded on daily basis. On day 35, all birds were euthanized to determine final body weight gain, feed intake and FCR.

Production Efficiency Index (PEI) was calculated according to the following formula:

$$Feed conversion ration \times Age of slaughter ( day )$$

$$PEI = Body weight ( kg ) \times Viability \times 100$$

Feed Cost per body weight gain (FCG) was calculated according to the following formula:

$$FCG = FCR \times Feed price ( USD\$ )$$

Carcass Traits and Meat Characteristic. Carcass and cut yields (abdominal fat, breast, wing, thigh, drumstick and back) were evaluated at the age of 35 days. The organs (heart, liver, proventriculus, gizzard, and jejunum) of one bird per pen (total n=8) were weighed and tissue of liver and jejunum were collected in a sealed bag and stored at −60° C.

Fatty liver assessment. Two birds per pen with body weight close to the pen average were selected and euthanized by a cardon dioxide chamber at the age of 35 days. The liver from each bird was extracted for fatty liver assessment using visual scoring.

Gut microbiome analysis. Ceca was collected from euthanized birds (one bird per pen) for four different bacterial counts: Lactobacillus spp., E. coli, Salmonella spp. and Clostridium perfringens.

TABLE 6
Effect of AW40 as a choline source on performance of broilers¹ (0-35 day)

| | | | Initial | Final | Body | | | | | |
|---|---|---|---|---|---|---|---|---|---|---|
| | | | body | body | weight | Feed | | | | Feed cost |
| | | Treatment | weight | weight | gain | intake | | Livability | Productior | per gain |
| Group | Diet | Added product | (g) | (g) | (g) | (g) | FCR² | (%) | index | (US$) |
| T1 | F1 | Control with MCL AW at 1.0 kg/T | 42 | 2391^{b} | 2349^{b} | 3195 | 1.360^{a} | 97.50 | 489.55 | 0.879^{a} |
| T2 | F1 | Choline Chloride with MCL | 42 | 2467^{a} | 2426^{a} | 3276 | 1.351^{ab} | 98.75 | 515.09 | 0.874^{ab} |
| | | AW at 1.0 kg/T | | | | | | | | |
| T3 | F1 | Choline Propionate with | 42 | 2411^{ab} | 2369^{ab} | 3182 | 1.343^{ab} | 97.50 | 500.39 | 0.868^{ab} |
| | | AW40 at 0.75 kg/T | | | | | | | | |
| T4 | F1 | Choline Propionate with | 42 | 2473^{a} | 2431^{a} | 3239 | 1.332^{b} | 95.00 | 503.23 | 8.861^{b} |
| | | AW40 at 1.0 kg/T | | | | | | | | |
| P-value | | | | 0.0420 | 0.0419 | 0.0808 | 0.0481 | 0.5046 | 0.2046 | 0.0418 |
| Pooled SEM | | | | 22.764 | 22.723 | 26.753 | 0.007 | 1.752 | 8.131 | 0.144 |
| C.V., % | | | | 2.64 | 2.68 | 2.35 | 1.38 | 5.10 | 4.58 | 1.39 |

^{a, b}Means within column with no common superscript differ significantly (P < 0.05).
¹Male ROSS308
²Feed conversion rate corrected for mortality and culls

Table 6 presented the growth performance of broilers in a choline deficient diet. Over the period of 35 days, feed supplementation with addition of AW40 at 1 kg/T (T4) showed significant reduction (P<0.05) in FCR (−0.028) compared to T1 control group and numerically better s compared to T2 (−0.019) and T3 (−0.011). T4 showed the best FCR, with significant reduction compared to T1 control and numerically better compared to T2 and T3. T4 demonstrated best Feed Cost per Body Weight (BW) Gain with significant reduction compared to T1 control (−US$0.018) and numerically better compared to T2 (−US$0.013) and T3 (−US$0.007). T3 performed numerically better than T1 control and T2 in terms of FCR. T3 also showed numerically lower Feed Cost per BW Gain than T1 control (−US$0.011) and T2 (−US$0.006).

TABLE 7
Effect of AW40 supplementation on carcass quality of broilers¹ (35 day)

| | | | | Breast | | Drum | | Abdominal |
|---|---|---|---|---|---|---|---|---|
| | | Treatment | Dressing² | meat² | Thigh² | stick² | Wing² | fat² |
| Group | Diet | Added product | (%) | (%) | (%) | (%) | (%) | (%) |
| T1 | F1 | Control with MCL AW at 1.0 | 75.82 | 30.45^{b} | 17.60^{a} | 13.43 | 9.43 | 1.37 |
| | | kg/T | | | | | | |
| T2 | F1 | Choline Chloride with MCL AW | 75.71 | 30.69^{b} | 17.57^{a} | | 9.51 | |
| | | at | | | | 13.46 | | 1.28 |
| | | 1.0 kg/T | | | | | | |
| T3 | F1 | Choline Propionate with | 75.98 | 31.33^{ab} | 17.10^{ab} | 13.56 | 9.58 | 1.29 |
| | | AW40 at 0.75 kg/T | | | | | | |
| T4 | F1 | Choline Propionate with | 76.47 | 32.40^{a} | 16.86^{b} | 13.10 | 9.42 | 1.30 |
| | | AW40 at 1.0 kg/T | | | | | | |
| P-value | | | 0.4354 | 0.0160 | 0.0217 | 0.2043 | 0.6429 | 0.8816 |
| Pooled SEM | | | 0.347 | 0.417 | 0.183 | 0.156 | 0.099 | 0.087 |
| C.V., % | | | 1.29 | 3.77 | 2.99 | 3.30 | 2.95 | 18.71 |

^{a, b}Means within column with no common superscript differ significantly (P < 0.05).
¹Male ROSS308
²% Carcass quality as % of carcass weight

Table 7 showed the carcass traits and cut yields at 35-day of age in the low choline diet. T4 Group has shown significantly higher breast meat percentage compared to T1 control and T2. However, T4 has significantly lower thigh % compared to control T1 and T2. T4 overall has the highest percentage of dressed meat (numerically) compared to other treatment groups.

TABLE 8
Effect of AW40 supplementation on organ weight and fatty
liver score of broilers¹ (35 day)

| | | | | | Proven- | | | |
|---|---|---|---|---|---|---|---|---|
| | | Treatment | Heart² | Liver² | triculus² | Gizzard² | Jejunum² | Fatty liver |
| Group | Diet | Added product | (%) | (%) | (%) | (%) | (%) | score³ 0-3 |
| T1 | F1 | Control with MCL AW at 1.0 | 0.37 | 2.54 | 0.32 | 1.12 | 1.29 | 0.44 |
| | | kg/T | | | | | | |
| T2 | F1 | Choline Chloride with MCL AW | 0.37 | 2.48 | 0.32 | 0.96 | 1.23 | 0.38 |
| | | at 1.0 kg/T | | | | | | |
| T3 | F1 | Choline Propionate with AW40 | 0.36 | 2.45 | 0.29 | 0.99 | 1.23 | 0.38 |
| | | at 0.75 kg/T | | | | | | |
| T4 | F1 | Choline Propionate with AW40 | 0.36 | 2.47 | 0.30 | 1.07 | 1.25 | 0.38 |
| | | at 1.0 kg/T | | | | | | |
| P-value | | | 0.9287 | 0.8526 | 0.2340 | 0.1649 | 0.8382 | 0.9840 |
| Pooled SEM | | | 0.009 | 0.077 | 0.011 | 0.053 | 0.048 | 0.137 |
| C.V., % | | | 6.85 | 8.82 | 10.34 | 14.41 | 10.95 | 27.91 |

¹Male ROSS308
²% Organ weight calculated as % of live weight
³Fatty liver score 0-3: Score 0: Normal liver (Dark red), Score 1: Mild case of fatty liver hemorrhagic syndrome (Mild yellow and hemorrhages), Score 2: Moderate case of fatty liver hemorrhagic syndrome (Light yellowish red liver and hemorrhages and Score 3: Extreme case of fatty liver hemorrhagic syndrome (Large and massive hemorrhages with putty colored liver)

Table 8 charted the organ weight percentage and fatty liver scores of the treatment groups for the low choline diet study. Fatty liver score was numerically higher in the T1 control group as expected due to the low choline content in feed but it was still within the normal range. There was no significant difference in the fatty liver scores for all treatment groups. Overall there was no difference in organ weight percentage for all treatment groups.

TABLE 9
Effect of AW40 as a choline source on cecal bacterial count of broilers¹ (35 d)

| | | | | | Clostridium | Salmonella |
|---|---|---|---|---|---|---|
| | | Treatment | Lactobacillus | E. coli | perfringens | spp. |
| Group | Diet | Added product | Log (CFU/g) | Log (CFU/g) | Log (CFU/g) | Log (CFU/g) |
| T1 | F1 | Control with MCL AW at 1.0 kg/T | 8.19 | 7.61 | 2.73 | 2.84 |
| T2 | F1 | Choline Chloride with MCL AW at | 8.20 | 7.54 | 2.70 | 2.76 |
| | | 1.0 kg/T | | | | |
| T3 | F1 | Choline Propionate with AW40 at 0.75 | 8.50 | 7.59 | 2.69 | 2.65 |
| | | kg/T | | | | |
| T4 | F1 | Choline Propionate with AW40 at 1.0 | 8.40 | 7.55 | 2.54 | 2.59 |
| | | kg/T | | | | |
| P-value | | | 0.0585 | 0.9642 | 0.7031 | 0.8670 |
| Pooled SEM | | | 0.090 | 0.114 | 0.123 | 0.228 |
| C.V., % | | | 3.06 | 4.26 | 13.07 | 23.83 |

Means within column with no common superscript differ significantly (P < 0.05).
¹Male ROSS 308

Table 9 showed the common gut microbiota count of the different treatment groups. Overall, there was no significant difference in cecal bacteria count among the different groups. T3 and T4 have numerically higher Lactobacillus spp count than T1 control and T2. T3 and T4 also have numerically lower Clostridium perfringens and Salmonella spp. CFU than T1 control and T2. Comparing the same amount of propionate content, T4 has numerically lower Clostridium perfringens count than T1 control (−0.19) and T2 (−0.16). T4 also showed numerically lower Salmonella spp. count than T1 control (−0.25) and T2 (−0.17).

### Study 2: Methionine Sparing Effect of AW40 on Performance of Broilers

TABLE 10
Effect of methionine sparing effect of AW40 on performance of broilers¹ (0-35 day)

| | | | Initial | Final | Body | | | | | |
|---|---|---|---|---|---|---|---|---|---|---|
| | | | body | body | weight | Feed | | | | Feed cost |
| | | Treatment | weight | weight | gain | intake | | Livability | Production | per gain |
| Group | Diet | Added product | (g) | (g) | (g) | (g) | FCR² | (%) | index | (US$) |
| T5 | F2 | Control with MCL AW at 1.0 | 42 | 2222 | 2180 | 3014^{b} | 1.383 | 95.00 | 436.12 | 0.910 |
| | | kg/T | | | | | | | | |
| T6 | F2 | Choline Chloride with MCL AW | 42 | 2287 | 2245 | 3051^{ab} | 1.359 | 96.25 | 463.14 | 0.896 |
| | | at 1.0 kg/T | | | | | | | | |
| T7 | F2 | Choline Propionate with AW40 | 42 | 2296 | 2254 | 3112^{a} | 1.381 | 100.00 | 475.25 | 0.909 |
| | | at 0.75 kg/T | | | | | | | | |
| T8 | F2 | Choline Propionate with AW40 | 42 | 2308 | 2266 | 3104^{a} | 1.370 | 98.75 | 475.59 | 0.902 |
| | | at 1.0 kg/T | | | | | | | | |
| P-value | | | | 0.0542 | 0.0535 | 0.0479 | 0.3163 | 0.3018 | 0.1078 | 0.3722 |
| Pooled SEM | | | | 22.230 | 22.211 | 26.144 | 0.010 | 2.004 | 12.246 | 0.215 |
| C.V., % | | | | 2.76 | 2.81 | 2.41 | 1.99 | 5.81 | 7.49 | 2.00 |

^{a, b}Means within column with no common superscript differ significantly (P < 0.05).
¹Male ROSS308
²Feed conversion rate corrected for mortality and culls

Growth performance results of broilers under the methionine deficient diet is summarized in Table 10. T6 performed best in terms of FCR and Feed Cost per Gain although it was not statistically significant compared to other treatment groups. T7 showed better Feed Cost per BW Gain compared to T5 control (−US$0.001). T8 showed numerically lower FCR compared to T5 Control and T7. T8 also demonstrated lower FCG compared to T5 control (−US$0.008) and numerically better compared to T7 (−US$0.007).

TABLE 11
Effect of methionine sparing effect of AW40 on carcass quality of broilers¹ (35 day)

| | | | | Breast | | Drum | | Abdominal |
|---|---|---|---|---|---|---|---|---|
| | | Treatment | Dressing | Meat² | Thigh² | stick² | Wing² | fat² |
| Group | Diet | Added product | (%) | (%) | (%) | (%) | (%) | (%) |
| T5 | F2 | Control with MCL AW at 1.0 kg/T | 73.60 | 28.27 | 17.65 | 14.13 | 10.01 | 1.25 |
| T6 | F2 | Choline Chloride with MCL AW at | 73.11 | 29.59 | 17.44 | 13.93 | 10.01 | 1.09 |
| | | 1.0 kg/T | | | | | | |
| T7 | F2 | Choline Propionate with AW40 at | 74.41 | 29.53 | 17.85 | 14.11 | 9.83 | 1.13 |
| | | 0.75 kg/T | | | | | | |
| T8 | F2 | Choline Propionate with AW40 at 1.0 | 74.06 | 29.15 | 17.45 | 13.98 | 9.79 | 1.16 |
| | | kg/T | | | | | | |
| P-value | | | 0.1182 | 0.1135 | 0.5465 | 0.9021 | 0.3464 | 0.6176 |
| Pooled SEM | | | 0.380 | 0.409 | 0.226 | 0.224 | 0.108 | 0.085 |
| C.V., % | | | 1.46 | 3.97 | 3.63 | 4.51 | 3.09 | 20.87 |

¹Male ROSS308
²% Carcass quality as % of carcass weight

Table 11 shows the carcass quality of the broilers in the methionine-deficient diet. Groups T7 and T8 containing choline propionate have numerically higher dressed meat percentage compared to T5 Control and T6. But overall, no significant difference was observed in the carcass quality for all treatment groups.

TABLE 12
Effect of methionine sparing effect of AW40 on organ weight and fatty liver score of
broilers¹ (35 day)

| | | | | | Proven- | | | Fatty liver |
|---|---|---|---|---|---|---|---|---|
| | | Treatment | Heart² | Liver² | triculus³ | Gizzard² | Jejunum² | Score³ |
| Group | Diet | Added product | (%) | (%) | (%) | (%) | (%) | 0-3 |
| T5 | F2 | Control with MCL AW at 1.0 kg/T | 0.41 | 2.64 | 0.33 | 1.21 | 1.32 | 0.69 |
| T6 | F2 | Choline Chloride with MCL AW at 1.0 | 0.41 | 2.48 | 0.34 | 1.12 | 1.28 | 0.44 |
| | | kg/T | | | | | | |
| T7 | F2 | Choline Propionate with AW40 at | 0.39 | 2.46 | 0.32 | 1.07 | 1.27 | 0.63 |
| | | 0.75 kg/T | | | | | | |
| T8 | F2 | Choline Propionate with AW40 at 1.0 | 0.41 | 2.47 | 0.36 | 1.13 | 1.32 | 0.63 |
| | | kg/T | | | | | | |
| P-value | | | 0.6105 | 0.1400 | 0.6338 | 0.2779 | 0.6908 | 0.5669 |
| Pooled SEM | | | 0.010 | 0.060 | 0.017 | 0.052 | 0.036 | 0.130 |
| C.V., % | | | 7.27 | 6.74 | 14.53 | 12.90 | 7.88 | 23.09 |

¹Male ROSS308
²% Organ weight as % of live weight
³Fatty liver score 0-3: Score 0: Normal liver (Dark red), Score 1: Mild case of fatty liver hemorrhagic syndrome (Mild yellow and hemorrhages), Score 2: Moderate case of fatty liver hemorrhagic syndrome (Light yellowish red liver and hemorrhages and Score 3: Extreme case of fatty liver hemorrhagic syndrome (Large and massive hemorrhages with putty colored liver)

Organ weights of broilers and fatty liver scores are tabulated in Table 12. Treatment groups, T6-T8 with higher amount of choline content demonstrated lower liver weight and better fatty liver score compared to T5 Control. Overall, there was no significant difference in organ weight percentage and fatty liver score for all treatment groups.

TABLE 13
Effect of methionine sparing effect of AW40 on cecal bacterial count of broilers¹ (35
day)

| | | | | | Clostridium | Salmonella |
|---|---|---|---|---|---|---|
| | | Treatment | Lactobacillus | E. coli | perfringens | spp. |
| Group | Diet | Added product | Log (CFU/g) | Log (CFU/g) | Log (CFU/g) | Log (CFU/g) |
| T5 | F2 | Control with MCL AW at 1.0 | 8.49 | 7.50 | 2.95 | 2.93 |
| | | kg/T | | | | |
| T6 | F2 | Choline chloride with MCL AW | 8.58 | 7.39 | 2.90 | 2.65 |
| | | at 1.0 kg/T | | | | |
| T7 | F2 | Choline Propionate with AW40 | 8.60 | 7.43 | 2.86 | 2.65 |
| | | at 0.75 kg/T | | | | |
| T8 | F2 | Choline Propionate with AW40 | 8.63 | 7.11 | 2.88 | 2.81 |
| | | at 1.0 kg/T | | | | |
| P-value | | | 0.9056 | 0.2048 | 0.9908 | 0.7569 |
| Pooled SEM | | | 0.139 | 0.131 | 0.206 | 0.213 |
| C.V., % | | | 4.59 | 5.04 | 20.10 | 21.88 |

Means within column with no common superscript differ significantly (P < 0.05).
¹Male ROSS308

Four types of cecal bacterial count is summarized in Table 13. Treatment Groups, T7 and T8 had numerically higher Lactobacillus spp CFU than T5 control and T6. T8 containing choline propionate has the lowest E. Coli and Clostridium perfringens CFU compared to all groups. When comparing the same amount of propionate content, T8 had numerically lower E. Coli count than T5 control (−0.39) and T6 (−0.28). Similarly, T8 had lower Clostridium perfringens CFU than T5 control (−0.07) and T6 (−0.12) given the same propionate content. T8 cecal also contained lower Salmonella spp. count than T1 control (−0.12). Overall, there was no significant difference in all the cecal bacterial count between the different treatment groups.

### Results

In Study 1, Treatment Group T4 with AW40 at 1.0 kg/T containing choline propionate reduced FCR and FCG, and demonstrated an increase in body weight gain. This proved that the choline propionate in AW40 could be metabolized and utilized by the broilers for better growth compared to treatment with choline chloride, in a low choline diet condition. Broilers in Treatment Group T4 showed the highest percentage of dressing breast meat among the studied groups and a significant increase (P<0.05) in breast meat compared to the T1 Control. Given its desirable white meat and health benefits, boneless chicken breast meat which is rich lean protein source, Vitamin D, Vitamin A, Vitamin C, Calcium and Iron is the most expensive cut of chicken in comparison to chicken thighs, wings, and drumsticks. It has been previously observed that fatty liver is usually evident in a diet deficient of choline. Treatment groups, T2, T3 and T4 with choline addition showed lower liver weight percentage and lower fatty liver score compared to T1 Control with no choline supplementation. However, the choline content in the F1 feed was likely not low enough to cause any significant visible fatty liver change.

In Study 2, supplementation of AW40 significantly increased feed intake with a tendency of improved body weight gain. FCR was not affected by lower level of AW40 supplementation (0.75 kg/T) but could be slightly improved at higher level of AW40 (1 kg/t) as compared to the T5 Control. Mortality was not affected by any product supplementation. Production index of birds fed methionine deficient diet could be increased and feed cost per gain could be reduced by AW40 or choline chloride supplementation. Supplementation of AW40 slightly improved dressing percentage and breast meat yield with some reduction in abdominal fat. Organ weight and fatty liver score were not significantly different among groups. However, there were trends of reduced liver weight, gizzard and fatty liver score in birds fed low methionine diet supplemented with choline chloride and AW40.

There were notable trends of reduced pathogenic bacteria (E. coli, Salmonella spp, Clostridium perfringens) and elevated counts of good bacteria (Lactobacillus spp.) in birds fed with diet supplemented with AW40.

Based on this study, supplementation of AW40 in feed diet as partial replacement of choline showed improvement in Feed Conversion Ratio (FCR) and Feed Cost per body weight Gain (FCG). This study further demonstrated that methionine sparing effect similar to that of by choline chloride could be achieved using AW40 containing choline propionate. In summary, the studies validated the viability of replacing ammonium hydroxide with choline hydroxide in the preparation of a mold inhibitor composition, where a mold inhibitor composition that contains choline propionate provides additional value with nutritional benefits on top of the mold inhibition function of commercially available products.

### Example 4

### Materials and Methods

The researchers investigated the use of choline hydroxide as a base to replace ammonium hydroxide as a neutralizing agent. The study was conducted with 288 layers at 50 weeks old. Birds were allocated into 4 treatments of experimental diet, each treatment having 6 replicates, with 3 cages (4 birds/cage) per replicate.

TABLE 14
Experimental design s
Trial 1

| | | | |
|---|---|---|---|
| No. of treatments | 4 | Birds per replicate | 12 |
| Replicates per treatment | 6 | Birds per treatment | 72 |
| Total No. of experimental unit | 24 | Total No. of birds | 288 |

The diets were provided to the hens at roughly 15-16% of crude protein. The hens were fed with the choline or choline-depleted diets (F1) for the initial 2 weeks for acclimatization to the new formulation. In this trial, a corn-SBM-DDGS based diet was formulated to include insufficient choline (no added choline) and used as basal Control Diet F1 (F1). The test was conducted by supplementing the diet with choline chloride, P1 (Myco CURB AW Liquid (MCL AW), which does not contain choline) and P2 (AW 40, containing 16% choline in the form of choline propionate). The treatments were added in the control basal diets in the mash mixing feed. Feed was prepared in crumble (mash) form for all birds until the completion of the 60-day test period.

Hens were fed the experimental diets for a period of 60 days. Water and feed were provided ad libitum or on demand. The provided and residual feed amount of each replicate was recorded daily and divided by the total egg mass (feed/egg mass, g/g). Egg production is expressed as average hen-day production, calculated from the total eggs divided by the total number of days.

### Egg Quality Analysis

From week 1 to 8, 30 eggs/group were collected each week for analyzing the egg quality. Eggshell thickness was measured by using a digital vernier caliper. Eggshell, albumin, and yolk weight were measured by using a digital scale. To determine another egg quality, the Digital Egg Tester DET 6000 (NABEL Co., Ltd., Kyoto, Japan) was utilized to evaluate the egg weight, eggshell strength, albumen height, Haugh unit (HU), and yolk color.

### Blood Serum Analysis

Blood samples were collected in vacutainer tubes through the brachial vein puncture. In accordance with the protocol of an aspartate aminotransferase (AST) Test Kit, the AST activity (IU/L) was determined by the kit using a BioMajesty® JCA-BM6010/C DiaSys Diagnostic Systems (Holzheim, Germany) with automated Chemistry Analyser BX-301 (Asia Green, Singapore).

A piece of breast muscle and abdominal fat (weighed about 250 mg) were homogenised in 2,500 μL of 10 mM phosphate-buffered saline (PBS) and then centrifuged at 1,600 g force for 10 minutes at 40° C. The supernatant was used directly in the protocol of a Cholesterol Test Kit (BioMajesty® JCA-BM6010/C: Cholesterol FS) with automated Chemistry Analyser BX-301 (Asia Green, Singapore). Cholesterol concentration (mg/dL) was calculated. The supernatant was also used in the Triglyceride Test Kit (BioMajesty® JCA-BM6010/C: Triglycerides FS) with automated Chemistry Analyser BX-301 (Asia Green, Singapore). Triglyceride concentration (mg/dL) was be calculated.

Eggs and liver of birds were collected at the end of the experiment and further analyzed for Total Lipids, and Fatty Acids. Total Lipids of eggs and liver were analysed by the Soxhlet method with a solvent extraction system (the Soxtec™ 8000, Foss Tecator AB, Sweden). For fatty acid composition analysis, in accordance with Folch et al. (1957), lipids from the experimental diet, eggs and liver samples were extracted with a chloroform-methanol (2:1) solution. Fatty acids were transformed into fatty acid methyl esters (FAME) using the method described by Morisson and Smith in 1964. The fatty acid content was determined using gas chromatography (GC) using the methodology described by Arjin et al. (2021). The gas chromatograph used for the experiment was the ShimadzuGC-2030, manufactured by Shimadzu in Kyoto, Japan. It was fitted with a Restek RT-2560 wall-coated fused wax capillary column, with dimensions of 0.25 mm×100 m×0.25 μm. The column was provided by Restek, located in Bellefonte, PA, USA. Helium served as the carrier gas. The temperatures of the injectors were maintained at 250° C. The oven temperature was set to increase at a rate of 3° C. per minute, starting at 100° C. and reaching 240° C. It then stayed at 240 degrees Celsius for a duration of 20 minutes. The 1 lL samples were introduced into the system, and the flame ionisation detector was adjusted to a temperature of 250° C. The identity of the samples was determined by comparing the retention durations of their peaks to those of FAME standard mixtures from Restek, located in Bellefonte, PA, USA.

After 60 days of intervention, 12 birds per treatments with body weight close to the pen average were selected and euthanized by a CO₂ chamber. The liver from each bird was taken for fatty liver assessment (visual scoring). Liver samples were blotted with tissue paper to remove blood and preserved in 10% neutral buffered formalin. Formalin-fixed liver samples were dehydrated in the series of ascending grades of alcohol (70%, 80%, 95%, and 100%), cleared in chloroform, impregnated with paraffin, and embedded in paraffin wax with ceresin. Paraffin blocks will were sectioned at 4 μm thickness using a sliding microtome. After sectioning, the sections were de-paraffinized in xylol followed by hydration in descending grades of alcohol (100%, 95%, 80%, and 70%) and distilled water. The sections will be stained with standard Haematoxylin and Eosin method and then mounted (DPX mountant).

### Results

The researchers observed that the addition of choline to the diet of laying hens resulted in increased egg production and greater overall egg mass. This improvement was observed when the animal diets were supplemented with choline at levels ranging between 0.75 and 1.0 kg/t.

In this study, the researchers observed that the addition of choline supplementation resulted in an increase in the amount of food consumed, which may have contributed to the improvement in egg production by increasing the availability of choline consumed.

The researchers further observed that P2 (AW 40) which included choline propionate was capable of imitating the hepatoprotective effects of choline. The results strongly suggest that the compositions of the present invention, specifically those containing an effective amount of choline propionate, showed significant improvements in productive performance parameters such as egg production and FCR. Additionally, P2 (AW 40) demonstrated hepatoprotective and lipotropic activity, as evidenced by the reduction in AST activity, improvement in liver histopathology, and decrease in lipid content in the eggs. These research findings validate that the compositions of the present invention have properties that make it a suitable alternative to compositions containing choline chloride that have been conventionally included in animal diets. The trial results are summarized in Tables 15-20.

TABLE 15
The effect of choline product supplementation on laying hens

| | Treatments | | | | | |
|---|---|---|---|---|---|---|
| Item | T1 | T2 | T3 | T4 | SEM | P-value |
| 1-4 weeks of experiment | | | | | | |
| Egg production (%) | 49.2^{b} | 87.7^{a} | 88.9^{a} | 89.9^{a} | 1.805 | <.0001 |
| ADFI (g) | 105.6^{b} | 117.4^{a} | 117.2^{a} | 118.1^{a} | 1.009 | <.0001 |
| Egg mass (g) | 29.9^{b} | 56.6^{a} | 56.6^{a} | 56.9^{a} | 1.348 | <.0001 |
| FCR | 3.03^{a} | 2.09^{b} | 2.19^{b} | 2.09^{b} | 0.194 | 0.0063 |
| 5-8 weeks of experiment | | | | | | |
| Egg production (%) | 17.8^{c} | 83.9^{b} | 87.7^{a} | 89.0^{a} | 0.932 | <.0001 |
| ADFI (g) | 93.8^{b} | 119.6^{a} | 119.0^{a} | 119.3^{a} | 1.104 | <.0001 |
| Egg mass (g) | 2.4^{c} | 51.8^{b} | 54.5^{a} | 55.3^{a} | 0.847 | <.0001 |
| FCR | 9.79^{a} | 2.31^{b} | 2.18^{b} | 2.17^{b} | 1.270 | 0.0014 |
| 1-8 weeks of experiment | | | | | | |
| Egg production (%) | 31.8^{c} | 85.6^{b} | 88.3^{ab} | 89.4^{a} | 1.087 | <.0001 |
| ADFI (g) | 99.1^{b} | 118.6^{a} | 118.2^{a} | 118.8^{a} | 0.805 | <.0001 |
| Egg mass (g) | 16.1^{b} | 54.2^{a} | 55.6^{a} | 56.1^{a} | 0.948 | <.0001 |
| FCR | 5.07^{a} | 2.21^{b} | 2.18^{b} | 2.13^{b} | 0.528 | 0.0014 |

ADFI, average daily feed intake;
FCR, feed conversion ratio (feed/egg mass, g/g).
^{a, b}means within the same row with different superscripts differ (P < 0.05).

TABLE 16
The effect of choline product supplementation on egg quality of laying hens

| | Treatments* | | | | | |
|---|---|---|---|---|---|---|
| Item | T1 | T2 | T3 | T4 | SEM | P-value |
| 1-4 weeks of experiment | | | | | | |
| Egg weight (g) | 61.0 | 61.9 | 61.6 | 60.7 | 0.560 | 0.4129 |
| Egg shell wt (g) | 6.81^{b} | 8.03^{a} | 7.87^{a} | 7.85^{a} | 0.109 | <.0001 |
| Yolk Weight (g) | 13.19^{b} | 13.28^{b} | 13.99^{a} | 13.83^{ab} | 0.218 | 0.0431 |
| Alb Weight (g) | 37.09 | 38.95 | 38.12 | 37.35 | 0.524 | 0.0843 |
| Strength (Kgf) | 2.56^{b} | 5.01^{a} | 4.85^{a} | 4.97^{a} | 0.134 | <.0001 |
| Shell-tnk (mm) | 0.232^{b} | 0.334^{a} | 0.328^{a} | 0.330^{a} | 0.005 | <.0001 |
| Alb height | 8.02 | 7.68 | 7.72 | 7.57 | 0.148 | 0.1963 |
| HU | 88.25 | 86.90 | 87.01 | 86.46 | 0.938 | 0.5795 |
| Yolk color | 10.59^{ab} | 10.28^{b} | 10.48^{c} | 10.75^{a} | 0.059 | 0.0001 |
| 5-8 weeks of experiment | | | | | | |
| Egg weight (g) | 58.2^{b} | 61.9^{a} | 63.2^{a} | 62.2^{a} | 0.526 | <.0001 |
| Egg shell wt (g) | 6.77^{b} | 9.04^{a} | 8.94^{a} | 9.00^{a} | 0.112 | <.0001 |
| Yolk Weight (g) | 16.21^{a} | 15.56^{b} | 16.29^{a} | 16.04^{a} | 0.139 | 0.0068 |
| Alb Weight (g) | 24.67^{b} | 37.30^{a} | 37.98^{a} | 37.17^{a} | 1.102 | <.0001 |
| Strength (Kgf) | 7.37 | 7.59 | 7.63 | 7.58 | 0.114 | 0.3892 |
| Shell-tnk (mm) | 0.218^{b} | 0.300^{a} | 0.295^{a} | 0.303^{a} | 0.004 | <.0001 |
| Alb height | 9.74 | 9.46 | 9.60 | 9.71 | 0.112 | 0.3106 |
| HU | 86.13^{ab} | 87.25^{a} | 86.03^{ab} | 84.86^{b} | 0.709 | 0.161 |
| Yolk color | 10.37^{b} | 10.40^{b} | 10.46^{b} | 10.61^{a} | 0.052 | 0.0172 |
| 1-8 weeks of experiment | | | | | | |
| Egg weight (g) | 59.7^{b} | 61.9^{a} | 62.5^{a} | 61.5^{a} | 0.418 | 0.001 |
| Egg shell wt (g) | 6.83^{b} | 8.59^{a} | 8.47^{ab} | 8.49^{a} | 0.078 | <.0001 |
| Yolk Weight (g) | 14.68^{b} | 14.55^{b} | 15.27^{a} | 15.06^{a} | 0.122 | 0.0016 |
| Alb Weight (g) | 30.57^{b} | 38.04^{a} | 38.04^{a} | 37.25^{a} | 0.599 | <.0001 |
| Strength (Kgf) | 4.93^{b} | 6.45^{a} | 6.39^{a} | 6.42^{a} | 0.137 | <.0001 |
| Shell-tnk (mm) | 0.225^{b} | 0.315^{a} | 0.310^{a} | 0.315^{a} | 0.003 | <.0001 |
| Alb height | 8.82 | 8.67 | 8.76 | 8.76 | 0.078 | 0.6119 |
| HU | 87.12 | 87.10 | 86.47 | 85.57 | 0.551 | 0.1913 |
| Yolk color | 10.46^{b} | 10.35^{b} | 10.47^{b} | 10.67^{a} | 0.039 | 0.0001 |

Egg shell wt, Egg shell weight;
Alb weight, Albumin weight;
Shell-tnk, Shell thickness;
Alb height, Albumin height;
HU, Haugh unit;
^{a, b}means within the same row with different superscripts differ (P < 0.05).

TABLE 17
The effect of choline product supplementation on serum
AST, cholesterol and Triglyceride content in
breast muscle and abdominal fat of laying hens

| | Treatments* | | | | | |
|---|---|---|---|---|---|---|
| Item | T1 | T2 | T3 | T4 | SEM | P-value |
| Serum | | | | | | |
| AST, U/L | 212^{a} | 239^{a} | 199^{ab} | 160^{b} | 14.5 | 0.0169 |
| Breast muscle | | | | | | |
| Cholesterol, | 6.5^{a} | 2.5^{b} | 3.5^{b} | 2.0^{b} | 0.92 | 0.0208 |
| mg/dL | | | | | | |
| Triglyceride, | 29.6^{a} | 15.5^{b} | 19.3^{b} | 14.8^{b} | 2.26 | 0.0019 |
| mg/dL | | | | | | |
| Abdominal fat | | | | | | |
| Cholesterol, | 1.50 | 2.25 | 1.50 | 1.75 | 0.27 | 0.2170 |
| mg/dL | | | | | | |
| Triglyceride, | 72.3 | 89.0 | 69.5 | 63.5 | 9.24 | 0.2917 |
| mg/dL | | | | | | |

*Feed Type F1 (Choline-deficient)
AST: Aspartate aminotransferase.
^{a, b}means within the same row with different superscripts differ (P < 0.05).

TABLE 18
The effect of choline product supplementation on total lipids in
eggs and livers from laying hens

| | Treatments* | | | | | |
|---|---|---|---|---|---|---|
| Item | T1 | T2 | T3 | T4 | SEM | P-value |
| Total lipids | | | | | | |
| (g/100 g) | | | | | | |
| Eggs | 61.31^{a} | 57.34^{a} | 51.43^{b} | 61.30^{a} | 1.058 | 0.0263 |
| Livers | 23.84 | 18.11 | 18.24 | 19.35 | 1.108 | 0.1609 |

*Feed Type F1 (Choline-deficient)
^{a, b}means within the same row with different superscripts differ (P < 0.05).

TABLE 19
The incidence of fatty liver in laying hens
fed with choline product supplementation

| | Treatments* | | | |
|---|---|---|---|---|
| Item | T1 | T2 | T3 | T4 |
| No. of sample observed | 108 | 108 | 108 | 108 |
| Rate for the fatty liver incidence (%) | 29.63 | 16.67 | 24.07 | 22.22 |

*Feed Type F1 (Choline-deficient)
^{a, b} means within the same row with different superscripts differ (P < 0.05).

TABLE 20
Effect of choline product supplementation on fatty
liver lesion severity in laying hens using Histopathology
Scoring with Hematoxylin and Eosin (H&E) Staining

| | Treatments* | | | | |
|---|---|---|---|---|---|
| Lesions | T1 | T2 | T3 | T4 | p-value |
| Liver | | | | | |
| Microvesicular | 0.5 (2-0) | 0 (0-0) | 0 (1-0) | 1.5 (2-0) | 0.06 |
| Macrovesicular | 0 (1-0) | 0 (0-0) | 0 (1-0) | 0 (2-0) | 0.07 |
| steatosis | | | | | |
| Hypertrophy | 0 (0.75-0) | 0 (1-0) | 0 (0.75-0) | 1 (2-0) | 0.15 |
| Ballooning | 0 (0-0) | 0 (0-0) | 0 (0-0) | 0 (0-0) | 0.39 |
| Fibrosis | 0 (0-0) | 0 (0-0) | 0 (0-0) | 0 (0-0) | 0.39 |
| Inflammation | | | | | |
| Number of | 1ª | 0.5^{ab} | 0.5^{ab} | 0^{b} | 0.05 |
| inflammatory foci | (1-0.25) | (1-0) | (1-0) | (0-0) | |
| Microgranulomas | 0 (0-0) | 0 (0-0) | 0 (0-0) | 0 (0-0) | 0.39 |
| Large | 0 (0-0) | 0 (0-0) | 0 (0-0) | 0 (0-0) | 0.29 |
| lipogranuloma | | | | | |
| Portal | 0.5 (1-0) | 0 (1-0) | 0.5 (1-0) | 0 (0-0) | 0.25 |
| inflammation | | | | | |

*Feed Type F1 (Choline-deficient)
^{a,b}median within the same row with different superscripts differ by Kruskal-Wallis test (P < 0.05).

As described herein, one aspect of the present invention relates to methods of supplementing an animal diet, for instance a poultry diet, with compositions containing choline propionate to improve or enhance animal performance or production.

In at least one embodiment, the animal diet is supplemented with a composition that contains an effective amount of choline propionate resulting in significantly improved the egg production and egg quality of laying hen, where supplementation of choline propriate in the layer diet resulted in layer performance similar to the diet 75% of choline in the form of choline chloride.

In another aspect of the present invention, the inventors have unexpectedly found that choline propionate is a suitable replacement for conventional choline supplementation compositions, for instance as a replacement of choline chloride, where the compositions of the present invention are capable of achieving comparable results to choline chloride supplementation in an animal diet, including when administered to the animal at a lower dosage, for instance in the range of about 10 ppm to 200 ppm in feed for choline content in the form of choline propionate.

Another aspect of the present invention relates to methods of controlling the growth of mold in animal feed and improving animal performance by adding a composition that contains choline propionate to the animal feed. Additionally, another aspect of the present invention relates to administering a choline salt, such as choline propionate, as a feed additive in an amount that inhibits mold in feed and provides additional nutritional benefit to the animal. In certain embodiments, the composition includes choline propionate in combination with other components, such as surfactants, co-surfactants, preservatives, pigments, colorants, flavoring, anti-microbial, antioxidant, or other additives.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. A feed additive comprising choline propionate in an amount sufficient to inhibit the growth of mold in the feed and improve animal performance compared to an animal diet that does not contain choline.

2. The feed additive of claim 1 where in the animal is a broiler or a laying hen.

3. The feed additive of claim 2 wherein the animal is a broiler and the animal performance is measured by body weight gain, feed intake, feed conversion ratio or production efficiency index.

4. The feed additive of claim 2 wherein the animal is a laying hen and the animal performance is measured by egg production, egg quality, yolk weight or yolk color.

5. The feed additive of claim 1 wherein the animal performance is the same or improved compared to the animal diet supplemented with choline chloride.

6. The feed additive of claim 1 wherein the animal performance is measured by a reduction of aspartate aminotransaferase activity.

7. The feed additive of claim 1 wherein the choline propionate is present in the feed in an amount ranging from about 10 ppm to about 200 ppm.

8. The feed additive of claim 1 further comprising at least one surfactant, co-surfactant, preservative, pigment, colorant, flavoring, anti-microbial, or antioxidant.

9. A method of improving animal performance comprising the step of adding a composition to an animal diet, where the composition contains choline propionate in an amount effective to improve the animal performance compared to an animal diet that does not contain choline.

10. The method of claim 9 wherein the animal is a broiler or a laying hen.

11. The method of claim 10 wherein the animal performance of the broiler is measured by body weight gain, feed intake, feed conversion ratio or production efficiency index.

12. The method of claim 10 wherein the animal performance of the laying hen is measured by egg production, egg quality, yolk weight or yolk color.

13. The method of claim 9 wherein the animal performance is the same or improved compared to the animal diet supplemented with choline chloride.

14. The method of claim 9 wherein the choline propionate is present in the feed in an amount ranging from about 10 ppm to about 200 ppm.

15. The method of claim 9 wherein the composition further comprises at least one surfactant, co-surfactant, preservative, pigment, colorant, flavoring, anti-microbial, or antioxidant.

16. The method of claim 9 wherein the animal performance is a protective liver effect measured by a reduction of aspartate aminotransaferase activity.

17. A process for preparing a mold inhibitor composition that contains choline propionate, where the process comprises the steps of using choline hydroxide as a base to neutralize propionic acid.

18. The process of claim 17 wherein the composition is added to animal feed and contains choline propionate in an amount effective to inhibit mold growth.

19. The process of claim 17 wherein the composition contains choline propionate in an amount ranging from about 10 ppm to about 200 ppm.

20. The process of claim 17 wherein the composition further comprises at least one surfactant, co-surfactant, preservative, pigment, colorant, flavoring, anti-microbial, or antioxidant.
